(19) United States
(12) Patent Application Publication
(10) Pub. No.: US 2003/0073155 A1
(43) Pub. Date: Apr. 17, 2003

(54) Methods for performing multiple diagnostic tests

(76) Inventors: Donald J. McMichael, South Jordan, UT (US); Kristy Peterson, Salt Lake City, UT (US); Barry J. Marshall, Dalkeith (AU)

Correspondence Address: KIMBERLY-CLARK WORLDWIDE, INC., 401 NORTH LAKE STREET, NEENAH, WI, 54956

(21) Appl. No.: 09/977,539

(22) Filed: Oct. 15, 2001

(51) Int. Cl. C12Q 1/04

(52) U.S. Cl. 435/34

(57) ABSTRACT

A method for diagnostic testing that includes obtaining a first specimen and, in some methods, obtaining a second specimen. The first specimen may be positioned in the first well of a carrier. The second specimen may be positioned in the second well of a carrier. The first well of the carrier may then be separated from the second well of the carrier.

The present invention relates generally to methods for performing multiple diagnostic tests.

In the medical arena, diagnostic testing is frequently performed to determine if a particular medical condition is present in a given patient. Diagnostic testing systems, which may be referred to as test kits, are manufactured to test for a wide variety of conditions in numerous types of biological test specimens, such as, for example, blood, tissue biopsies, and saliva. Such testing systems may be utilized to determine the presence of particular bacteria, such as Helicobacter pylori. Some tests that have been proposed to detect Helicobacter pylori include those that are disclosed in numerous U.S. Patents, including, for example, U.S. Pat. No. 4,748,113 to Marshall, U.S. Pat. No. 5,314,804 to Boguslaski et al., U.S. Pat. No. 5,439,801 to Jackson, U.S. Pat. No. 5,702,911 to Whalen, U.S. Pat. No. 5,989,840 to D'Angelo et al., U.S. Pat. No. 6,068,985 to Cripps et al., U.S. Pat. No. 6,156,346 to Chen et al., and U.S. Pat. No. 6,187,556 to Lee et al., each of such patents being incorporated in their entirety by reference herein.

The present invention includes a method for diagnostic testing which includes obtaining a first specimen and obtaining a second specimen. The first specimen is positioned in a first well of a carrier, and a second specimen is positioned in a second well of a carrier. The first well of the carrier is separated from the second well of the carrier.

The first specimen may also be subjected to a test, and, in some embodiments, that test may detect the presence of Helicobacter pylori and be disposed within the first well. The second specimen may also be subjected to a test, and, in some embodiments, that test may detect the presence of Helicobacter pylori and be disposed within the second well. In selected embodiments, the second specimen may be preserved for use in a subsequent test.

Various embodiments of the present invention may utilize a carrier having a first well and a second well. The carrier may also include a separator that permits the first well to be separated from the second well. The separator may be configured as an indentation, one or more perforations, or a depression formed in any surface or structure of the carrier.

A specimen-handling tool may also be used with the carrier. In some embodiments, the specimen-handling tool may be disposed about at least a portion of one of the first and/or second wells. Selected embodiments may include an overlying member that is positioned adjacent to the carrier so that the overlying member is disposed over at least a portion of one of the first or second wells. A plug may be disposed in at least one of the wells, the plug being attached to the overlying member so that, when the overlying member is removed from the carrier, the plug is removed from the well.

The specimen-handling tool may include a pair of cooperating arms. Each arm of the specimen handling tool may include a tip portion and a rear portion, the arms being joined to each other at their rear portions to form a joined end. The tip portions may be variously formed, and may be formed as a flat surface, a point or a fork. Each arm may also include a rearward arcuate portion, a forward arcuate portion, and an intermediate arcuate portion, the intermediate arcuate portion being disposed between the rearward arcuate portion and the forward arcuate portion. The arcuate portions may be configured so that the area disposed between the pair of arms is substantially hourglass in shape.

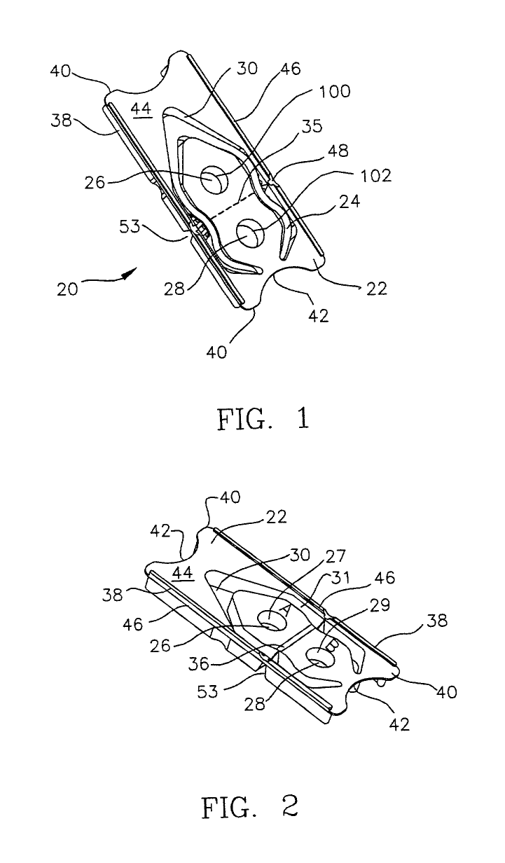

FIG. 1 is a perspective view of an embodiment of the system, carrier and specimen-handling tool of the present invention.

FIG. 2 is a perspective view of an embodiment of the carrier of the present invention.

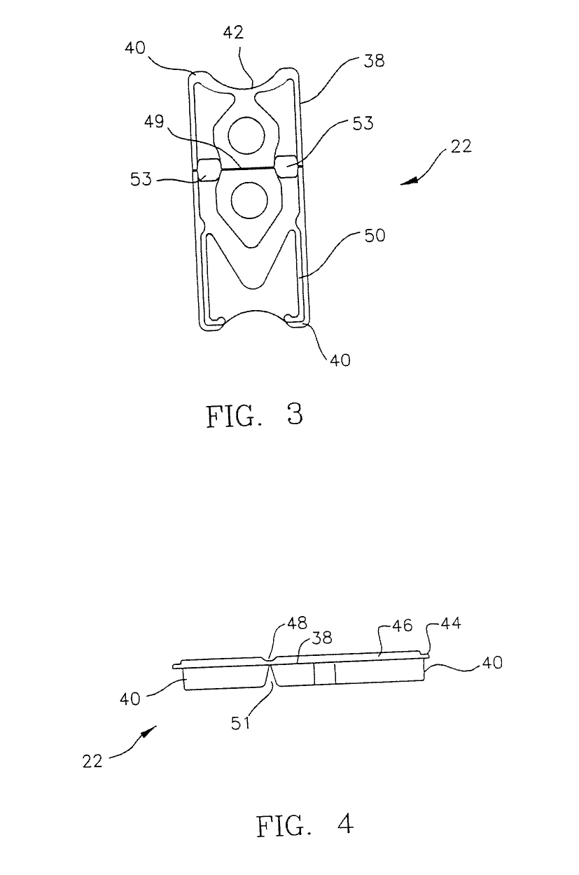

FIG. 3 is a view of the bottom of an embodiment of the carrier of the present invention.

FIG. 4 is a side view of an embodiment of the carrier of the present invention.

FIG. 5 is a top view of another embodiment of the carrier of the present invention.

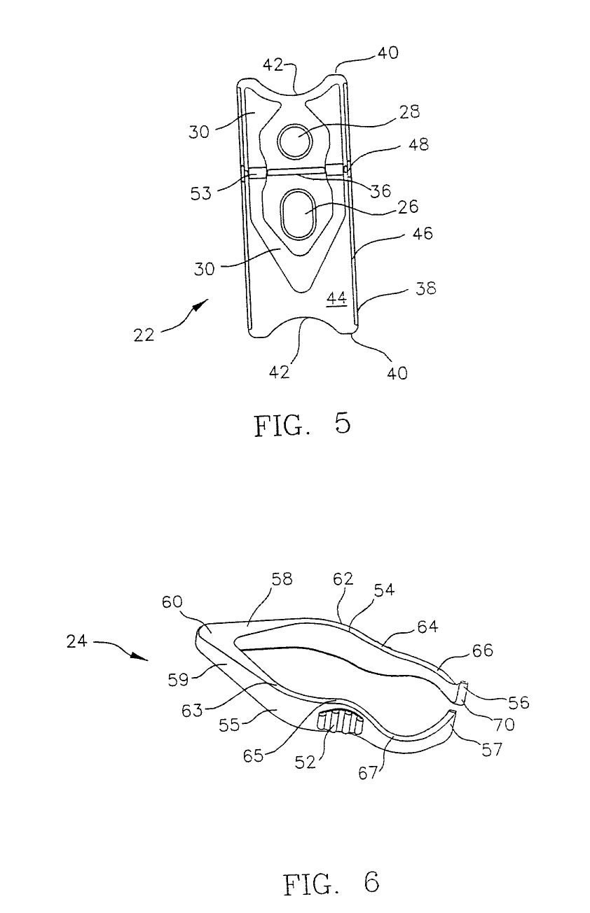

FIG. 6 is a perspective view of an embodiment of the specimen-handling tool of the present invention.

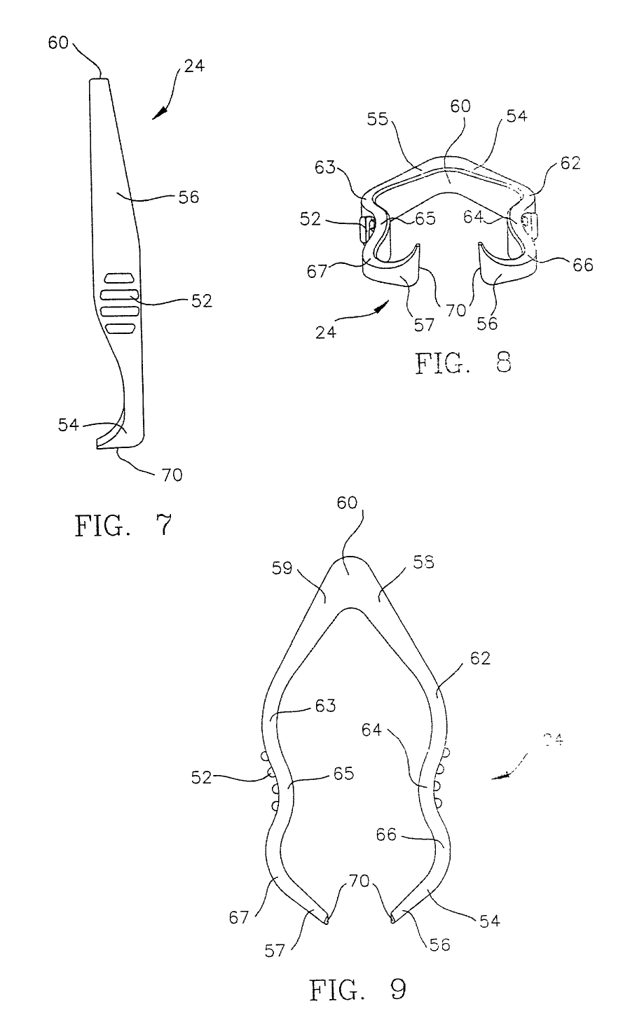

FIG. 7 is a side view of an embodiment of the specimen-handling tool of the present invention depicted in FIG. 6.

FIG. 8 is another perspective view of an embodiment of the specimen-handling tool of the present invention.

FIG. 9 is a top view of the embodiment of the specimen-handling tool of the present invention that is depicted in FIG. 8.

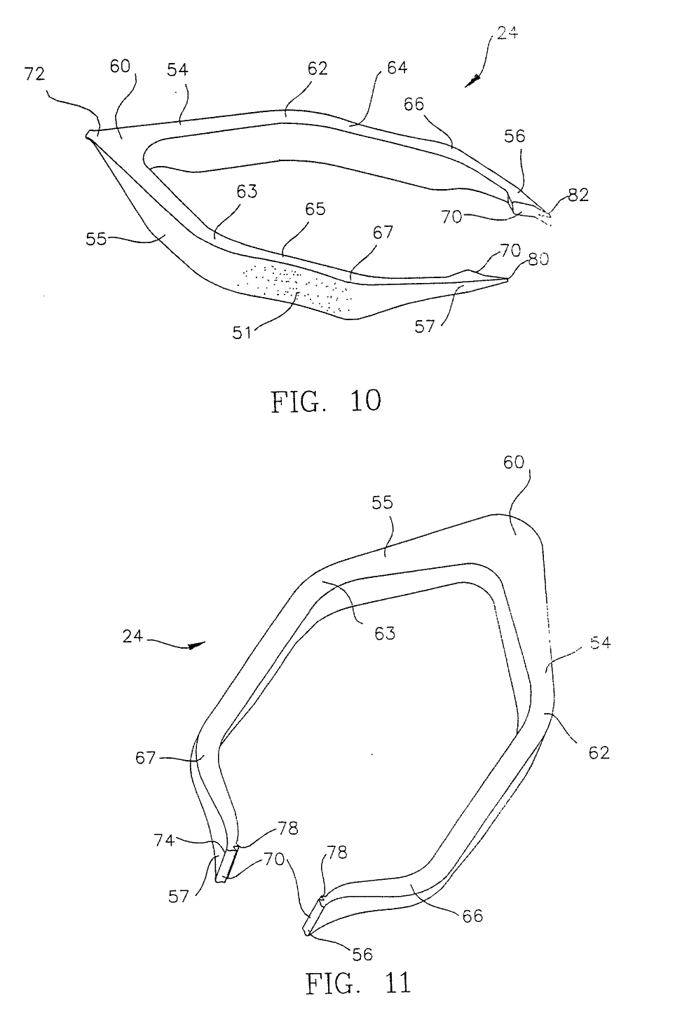

FIG. 10 is a perspective view of yet another embodiment of the specimen-handling tool of the present invention.

FIG. 11 is a perspective view of still another embodiment of the specimen-handling tool of the present invention.

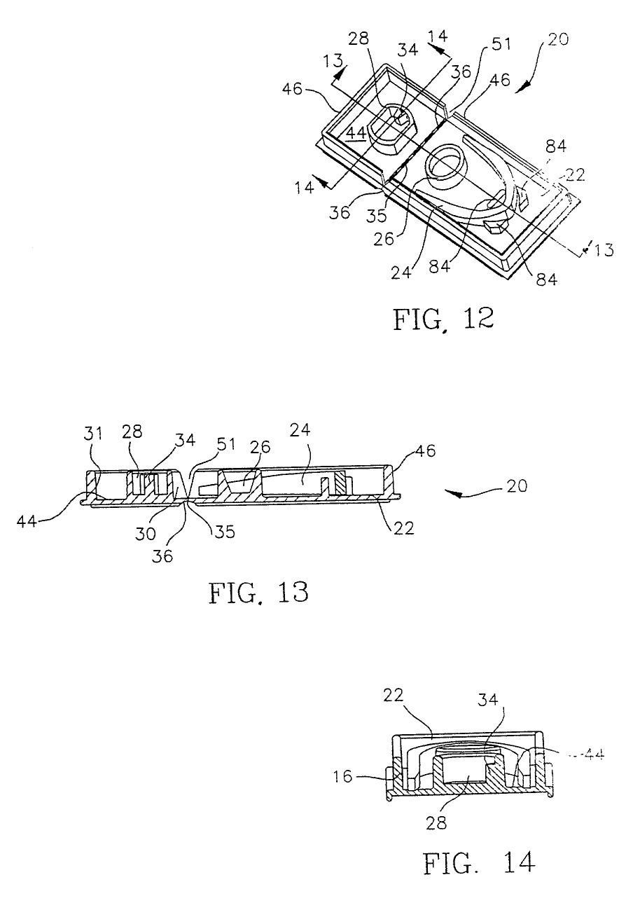

FIG. 12 is a perspective view of another embodiment of the system, carrier and specimen-handling tool of the present invention.

FIG. 13 is a cross-sectional view of the embodiment depicted in FIG. 12, taken along line 13-13.

FIG. 14 is a perspective cross-sectional view of the embodiment depicted in FIG. 12, taken along line 14-14.

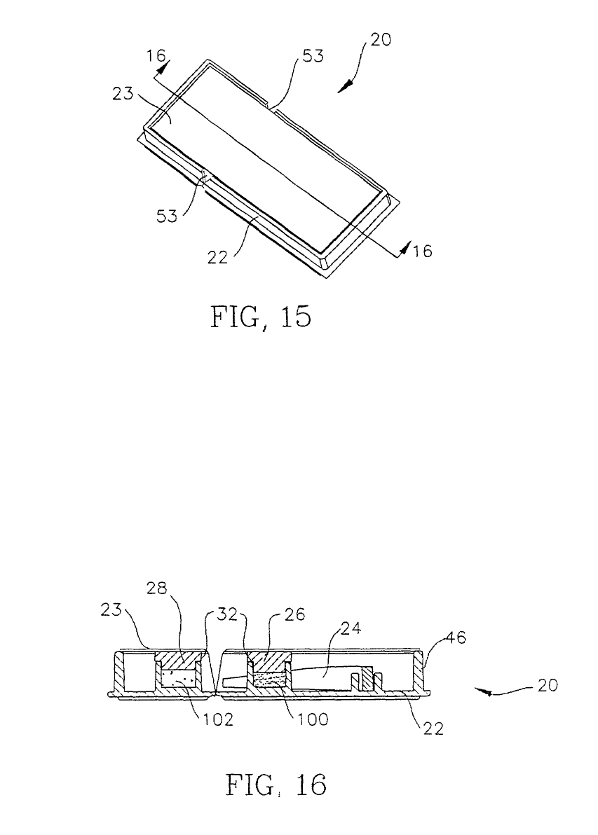

FIG. 15 is a perspective view of another embodiment of the system of the present invention.

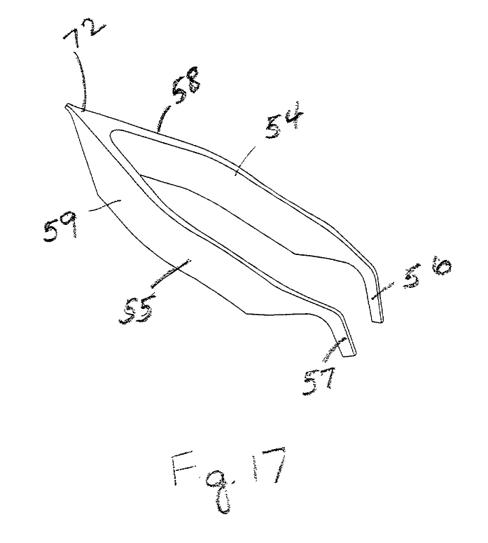

FIG. 16 is a cross-sectional view of the embodiment depicted in FIG. 15, taken along line 16-16.

FIG. 17 is a perspective view of another embodiment of the specimen-handling tool of the present invention.

FIG. 1 discloses an embodiment of a diagnostic system 20 according to the present invention that may be utilized for many types of diagnostic testing.

Such diagnostic tests utilize a biological test specimen such as, for example, tissue biopsy, blood or saliva. The diagnostic system 20 may include a carrier 22 and a mechanism by which a user may manipulate a sample of tissue, such as, for example, the specimen-handling tool 24 that is shown in FIGS. 1, 6 and 10. As depicted in FIG. 15, the diagnostic system 20 may further include an overlying member 23.

As shown in FIGS. 1-3, 5, and 12, the carrier 22 may include a first well 26 and a second well 28. The wells 26 and 28 may be defined, at least in part, by the walls 27 and 29, respectively. The wells 26 and 28 may be formed to have a variety of different depths and cross-sectional shapes, some variations of which are shown in FIGS. 5, 12-14 and 16. The wells 26 and 28 of the carrier 22 may be variously formed, and may have similar configurations or dissimilar configurations. As shown in FIGS. 1, 2, and 5, the wells 26 and/or 28 are generally frustoconical in shape, although the wells 26 and/or 28 may be cylindrical or otherwise shaped. The wells 26 and/or 28 may be formed so that, when viewed from the top of the carrier 22, the wells 26 and/or 28 have a non-circular shape, such as an elliptical, square, rectangular, D-shaped or any other shape.

One or more projecting members, such as the projecting member 34 that is shown in FIGS. 12-14, may be disposed within one or both of the wells 26 and 28. At least a portion of the projecting member 34 may be disposed outside of the interior of the wells 26 and/or 28. The projecting member 34 may be integrally formed with the walls 27 and 29, or may be attached to the walls 27 and/or 29. Such projecting members 34 may be configured to assist removal of the specimen such as, for example, a biopsy specimen, from the specimen-handling tool 24. These projecting members 34 may be configured to assist the user in accurately positioning a specimen within the well 26 or 28.

The wells 26 and 28 may also include a step such as the step 32 that is depicted in FIG. 13.

The carrier 22 may have many different overall exterior shapes, such as, for example, the generally rectangular shape as shown in FIGS. 1, 2 and 5. The carrier 22 may be alternately shaped, such as, for example, square, oblong, triangular, and the like. The carrier 22 may, as shown in FIGS. 1-3, include two elongated sides 38, two ends 40 and a surface 44. The ends 40 may be configured to be easily grasped by a user and one, none or both of the ends 40 may include an arcuate portion 42 as shown in FIGS. 1-5.

As shown in FIGS. 1, 2, 4 and 5, the carrier 22 may include a surface 44. The first and/or second wells 26 and 28, respectively, may be configured to extend downwardly from the surface 44. As shown in FIGS. 1 and 2, the carrier 22 may also include a cavity 30. In a similar manner, the cavity 30 may be configured to extend downwardly from the surface 44, as shown in FIGS. 1, 2 and 5. As shown in FIGS. 12-14, one or both of the wells 26 and 28 and/or the cavity 30 may be formed so as to extend upwardly from at least a portion of the surface 44.

A mechanism by which a user may manipulate a sample of tissue, such as, for example, the specimen handling tool 24 such as that shown in FIGS. 1 and 6-11, may also be included in particular embodiments of the diagnostic system 20 of the present invention. The specimen-handling tool 24 may be disposed within the cavity 30.

The cavity 30 may, as shown in FIGS. 1-3, be configured so that it is disposed about at least a portion of one of the first and/or second wells 26 and 28, respectively. The carrier 22 may also be configured so that a specimen handling tool 24 may be otherwise retained in the carrier 22 so that it is disposed about at least a portion of one of the first and/or second wells 26 and 28, respectively. As shown in FIGS. 12 and 13, the carrier 22 may be configured so that the specimen-handling tool 24 is secured in a particular position by one or more ribs 84. The specimen-handling tool 24 may be removably attached to the carrier 22 by one or more locking arms, breakaway tabs, adhesive, or the like.

One or more rails 46 may be included in selected embodiments of the present invention and may be disposed on the carrier 22 so that the rails extend upwardly along at least a portion of the surface 44. One or more rails 46 may also be configured to extend outwardly from the carrier 22. At least one gap 48 may be formed in one of the rails 46 that extend along a portion of the carrier 22.

As shown in FIG. 3, one or more supports 50 may be provided which extend downwardly from the surface 44. As seen in FIG. 3, the supports 50 may be attached to the wall (or walls) 31 that form at least a portion of the cavity 30 and may extend outwardly from those wall 31 to permit the carrier 22 to rest in a stable position on a horizontal or other surface. The rails 46 and the supports 50 may be configured to enable the carrier 22 to be automatically processed through a variety of equipment.

If desired, the surface 44 may be configured so that various indicia, such as letters, numbers, symbols and other characters, may be placed onto or formed into the surface 44. For example, and as shown in FIG. 2, each well 26 and/or 28 may be given a particular designation, such as A or B, and that designation may be printed upon the surface 44.

The carrier 22 may be formed from a variety of materials, including, for example, polycarbonate, polystyrene, polypropylene, polyethylene, polyvinylchloride, or any other type of polyolefin.

A separator may be disposed between the first and second wells 26 and 28, respectively, to permit the first well 26 to be separated from the second well 28. For example and as shown in FIG. 1, the separator may be configured as a series of perforations 35 which are configured to permit the carrier 22 to be broken into two separate portions; a first portion containing the first well 26 and a second portion containing the second well 28. The separator may also include a single perforation 35, as shown in FIG. 12. The gaps 48 in the rails 46 may be positioned to enhance the separability of the wells 26 and 28 from each other, as seen in the embodiment depicted in FIG. 1.

As shown in FIG. 2, the separator may also be formed as or include a depression 36, which may be formed in the surface 44 of the carrier 22. The depression 36 may have many different shapes, such as, for example, v-shaped or arcuate.

As seen in FIGS. 3 and 4, the separator may also include one or more notches 53 that are formed in the carrier 22. The notches 53 may be formed in the cavity 30 and may be used to enhance the separability of the carrier 22. As also shown in FIG. 3, an indentation 49 may be formed on the underside of the carrier 22. The indentation 49 may be variously configured, and may be v-shaped. As seen in the embodiment depicted in FIG. 3, the indentation 49 may extend across substantially the entire width of the carrier 22.

Any of the structures disclosed herein may be used alone or in combination with each other to form the separator of the present invention. For example and as shown in FIG. 12, a perforation 35 may be positioned within a depression 36 that is disposed on the surface 44 of the carrier 22. In the same embodiment, a pair of notches 53 may be positioned on the carrier 22 to assist in separating the first well 26 from the second well 28.

A wide variety of compounds may be disposed within the first and/or second wells that permit the testing of a specimen such as, for example, a tissue biopsy specimen. In some embodiments, compounds such as those described in the patents listed herein may be used in the present invention to test for Helicobacter pylori.

The ability to separate the first well from the second well can be beneficial to users of such a test system. For example, in a particular embodiment, a composition which tests a specimen for a particular bacteria may be disposed in the first well 26 while the second well 28 may contain a composition which tests for a different bacteria. The tests may be separated from each other before or after the insertion of specimens into the wells 26 and 28. Such a feature may assist in processing, monitoring, handling or storage of the tests.

In some embodiments, the well 28 may contain a medium such as an agar that preserves a specimen. In such embodiments, if it is desired or necessary to repeat the analysis performed in the first well 26, it is not necessary to obtain another specimen, as the specimen contained within the second well 28 may be subjected to the particular test when desired. In such a situation, the specimen that is retained within the second well 28 may be subjected to different environmental conditions to assist in preserving the specimen while the first well 26 may be subjected to different environmental conditions to assist in obtaining expedited results.

Of course, any composition may be disposed in either of the wells 26 or 28, and it is not required that any particular composition be disposed within the first well 26.

In such an embodiment, a method for diagnostic testing may be utilized which includes the steps of obtaining a first specimen and, in some methods, obtaining a second specimen. The specimen may, in some instances, be a biological specimen such as a tissue biopsy specimen.

The method may further include providing a carrier 22 which has a first well 26, a second well 28, and a specimen-handling tool 24 that may be disposed within at least a portion of the carrier 22. Additionally, the carrier may include a separator disposed between the first well and the second well, the separator adapted to permit the separation of the first well and the second well.

A composition 100 may be provided within the first well 26 that is adapted to detect the presence of Helicobacter pylori. A composition 102 may also be provided within the second well 28, the composition 102 being adapted to detect the presence of Helicobacter pylori.

The first specimen may be disposed or positioned in the first well 26 of the carrier 22. The second specimen may be disposed or positioned in the second well 28 of the carrier 22. In some methods, the specimens may be positioned within the first well 26 or the second well 28 by using a specimen-handling tool 24.

The first well 26 may be separated from the second well 28 before or after placing the specimens within the first well 26 and the second well 28. As discussed above, the first specimen and/or the second specimen may be subjected to a test by placing any of a wide variety of testing compositions within the first well 26 and/or the second well 28. The first and second specimens may be subjected to different types of tests. Additionally, the second specimen may be preserved for use in a subsequent test.

Particular embodiments of the specimen-handling tool 24 are shown in FIGS. 6-11 and 17. The specimen-handling tool 24 may include, as shown in FIGS. 6-9, a pair of cooperating arms 54 and 55. Each arm 54 and 55 may include a tip portion 56 and 57, respectively. The arms 54 and 55 may each also include a rear portion 58 and 59, respectively. The arms 54 and 55 may be joined to each other at their rear portions 58 and 59, respectively, forming a joined end 60. The joined end 60 may be configured to assist the user in accomplishing particular tasks, such as, for example, manipulating a specimen, removing a plug 86 (see FIG. 14) from one of the first and/or second wells 26 and 28, respectively, as well as other tasks. The outermost portion of the joined end 60 may be variously configured, and may be formed as a narrow projection, such as that shown in FIG. 10.

As seen in FIGS. 8 and 9, each arm 54 and 55 may also include a rearward arcuate portion 62 and 63, respectively, and a forward arcuate portion 66 and 67, respectively. Disposed between each rearward arcuate portion 62 and 63 and its corresponding forward arcuate portion 66 and 67, respectively, is an intermediate arcuate portion 64 and 65, respectively. The arcuate portions 62-64-66 and 63-65-67 of each arm 54 and 55, respectively, may be configured so that the area disposed between the arms 54 and 55 is approximately hourglass in shape. In such an embodiment, the rearward arcuate portions 62 and 63 and forward arcuate portions 66 and 67 curve outwardly, and the intermediate arcuate portions 64 and 65 curve inwardly.

The intermediate arcuate portions 64 and 65 may be formed so that a user may more easily grip these portions. As shown in FIG. 6, one or more ribs 52 may be positioned on the outer surface of the intermediate arcuate portions 64 and 65. Alternately, a portion of the arms 54 and/or 55 may have a roughened texture to enable a user to more effectively grasp and manipulate the specimen-handling tool 24, such as is shown in FIG. 10 at 51.

The arms 54 and/or 55 may include fewer or more arcuate portions than the three arcuate portions described above, such as the specimen-handling tool shown in FIG. 11. The arcuate portions of the arms 54 and/or 55 may have a more or less pronounced arcuate shape than what is depicted in FIG. 6. For example and as shown in FIGS. 10-12 and 17, other configurations of the arms 54 and 55 may be used in the specimen-handling tool 24.

The tip portions 56 and 57 may be variously formed to enable a user to manipulate a specimen. The tip portions 56 and 57 may be formed to include a surface such as the surfaces 70. The surfaces 70 may be variously shaped and, in particular, one or both of the surfaces 70 may be curved (as shown in FIG. 10) or flat (as shown in FIG. 6). The surfaces 70 may be rough or smooth. Also, structures such as the ridges 78 that are depicted in FIG. 11 may also be positioned on one or more of the surfaces 70. The surfaces 70 may be disposed so that they are at least somewhat facing each other, thereby enabling a user to grasp a specimen and hold it between the surfaces 70. As shown in FIG. 10, the tip portions 56 and/or 57 may curve outwardly, and may, in some embodiments such as is shown in FIG. 11, end in a relatively sharp edge 74. One or both of the tip portions 56 and 57 may include a point, such as the point 80 shown in FIG. 10 or a fork 82, also shown in FIG. 10, or any number of other configurations.

The specimen-handling tool may be formed from a variety of materials, including, for example, plastics including polycarbonate, polystyrene, polypropylene, polyethylene, polyvinylchloride, or any other type of polyolefin.

Referring now to FIGS. 15 and 16, an overlying member 23 may be disposed over at least a portion of the surface 44 of the carrier 22. At least a portion of the cavity 30 may be formed by the wall 31. The overlying member 23 may take the form of an adhesive-backed label that adheres to at least a portion of the surface 44. The overlying member 23 may overly any combination of the first well 26, the second well 28 and the cavity 30.

The overlying member 23 may also be used to seal the first and second wells 26 and 28, respectively. In some embodiments, the overlying member may be used to regulate the rate of water vapor transmission to and from the wells 26 and 28 of the carrier 22. The overlying member 23 may also be configured so that, if the overlying member 23 is removed prematurely or inadvertently, it may be easily reapplied to the carrier 22 so that the wells 26 and 28 may be resealed.

The overlying member 23 may also be used to retain the specimen-handling tool 24 within the cavity 30. The overlying member 23 may also be configured only to retain the specimen-handling tool 24 within the cavity 30. In some embodiments, the overlying member 23 may be adhered to at least a portion of the specimen-handling tool 24 so that, when the overlying member 23 is removed form the carrier 22, the specimen-handling tool 24 is also removed from the carrier 22. Although this may be accomplished in many different ways, the intermediate arcuate portions 64 and 65 may, when the specimen-handling tool 24 is positioned within the cavity 30, be level with or rise slightly above the surface 44 so as to contact and be adhered to the overlying member 23.

In some embodiments, the overlying member 23 may also be configured to separate into two distinct portions so that, when the first well 26 is separated from the second well 28, the overlying member 23 may also be separated and used to cover the first well 26 and the second well 28.

As shown in FIG. 16, a plug 86 may also be used to at least partially seal each well 26 and 28. In such a configuration, the overlying member 23 does not need to seal the well that contains the plug 86, but may merely be positioned above the well 26 and/or 28. The plug 86 may be formed from a variety of materials, including, for example, rubber, wax, silicone, or any of a variety of plastics. In some embodiments, a film cover 86, shown in FIG. 14, may also be applied to a portion of the carrier 22, such as, for example, the well 28.

In some embodiments, the overlying member 23 may be adhered or otherwise connected to one or more of the plugs 86 so that, when the overlying member 23 is separated from the carrier 22, one or more of the plugs 86 may also be removed. The plug 86 may also be removed with the specimen-handling tool.

The invention may be embodied in other specific forms without departing from the scope and spirit of the inventive characteristics thereof. The present embodiments therefore are to be considered in all respects as illustrative and not restrictive, the scope of the invention being indicated by the appended claims rather than by the foregoing description, and all changes which come within the meaning and range of equivalency of the claims are therefore intended to be embraced therein.

It is emphasized that the Abstract is provided to comply with the rules requiring an abstract that will allow a searcher or other reader to quickly ascertain the subject matter of the technical disclosure. It is submitted with the understanding that it will not be used to interpret or limit the scope or meaning of the claims. 37 CFR 1.72(b).

We claim:

1. A method for diagnostic testing comprising:
obtaining a first specimen;
obtaining a second specimen;
positioning the first specimen in a first well of a carrier;
positioning the second specimen in a second well of a carrier; and
separating the first well of the carrier from the second well of the carrier.

2. The method of claim 1 further comprising subjecting the first specimen to a test.

3. The method of claim 1 further comprising subjecting the second specimen to a test.

4. The method of claim 1 further comprising preserving the second specimen for use in a subsequent test.

5. The method of claim 1 further comprising providing a carrier having a first well, a second well, and a specimen-handling tool disposed within at least a portion of the carrier.

6. The method of claim 1 further comprising providing a carrier having a separator disposed between the first well and the second well, the separator adapted to permit the separation of the first well and the second well.

7. The method of claim 1 further comprising providing a composition adapted to detect the presence of Helicobacter pylori in the first well.

8. The method of claim 7 further comprising providing a composition adapted to detect the presence of Helicobacter pylori in the second well.

9. The method of claim 1 further comprising providing a composition adapted to detect the presence of Helicobacter pylori in the first well.

10. A method for diagnostic testing comprising:
providing a carrier having a first well, a second well separable from the first well, and a specimen-handling tool disposed within at least a portion of the carrier;
obtaining a first specimen;
obtaining a second specimen;
positioning the first specimen in the first well of the carrier with the specimen-handling tool;
positioning the second specimen in the second well of the carrier with the specimen-handling tool; and
separating the first well of the carrier from the second well of the carrier.

11. The method of claim 10 further comprising subjecting the first specimen to a test to determine the presence of Helicobacter pylori.

12. The method of claim 10 further comprising subjecting the second specimen to a test to determine the presence of Helicobacter pylori.

13. The method of claim 10 further comprising preserving the second specimen for use in a subsequent test.

14. A method for conducting diagnostic testing comprising:
obtaining a first biopsy specimen and a second biopsy specimen;
disposing the first biopsy specimen in a first well of a carrier;
disposing the second biopsy specimen in a second well of a carrier; and
separating the first well of the carrier from the second well of the carrier.

15. The method of claim 14 further comprising subjecting the first specimen to a test to determine the presence of Helicobacter pylori.

16. The method of claim 14 further comprising subjecting the second specimen to a test to determine the presence of Helicobacter pylori.

17. The method of claim 14 further comprising preserving the second specimen for use in a subsequent test.
